(19) United States
(12) Patent Application Publication
(10) Pub. No.: US 2023/0197215 A1
(43) Pub. Date: Jun. 22, 2023

(54) System and Related Methods for Rapid Identification and Verification of Immunization Status

(71) Applicant: MATTHEW CHARLES JAMES, FAYETTEVILLE, GA (US)

(72) Inventor: MATTHEW CHARLES JAMES, FAYETTEVILLE, GA (US)

(21) Appl. No.: 17/509,197

(22) Filed: Oct. 25, 2021

(51) Int. Cl. G16H 10/60; G06F 21/32

(52) U.S. Cl. CPC G16H 10/60; G06F 21/32; G06K 7/10297

(57) ABSTRACT

The present disclosure provides a system and related methods for automatically recording and managing an immunization record for one or more animals, storing the records on an immutable distributed ledger or blockchain and thus facilitating rapid verification of the status of an animal prior to travel or entry into a restricted area. This allows for immediate on-demand determination of whether pets, wildlife, and livestock are in compliance with the necessary requirements for entry or travel.

### FIELD OF INVENTION

The present invention relates generally to generally to methods and systems for managing and updating an immunization database. More specifically, the present invention relates to a system comprising a distributed ledger for recording and storing immunization credentials for one or more animals.

### BACKGROUND

The need for secure methods and systems for tracking and authenticating immunization status of both people and animals has become very apparent following the COVID-19 global pandemic, particularly for travel-related industries. Currently the systems in place for achieving this are both archaic and inefficient, relying heavily on printed documents which can be easily falsified, lost, and transcribed incorrectly.

Companies and individuals that commonly need to transport pets, wildlife and livestock across borders in particular encounter such issues on a regular basis, and go through a great deal of trouble and frustration as a result of lost records, bad actors falsifying records, and the ever-changing legal requirements, both domestically and internationally. Failing to stay on top of and meet these legal requirements can also result in facing costly penalties and fines.

For example, the US government has recently implemented the H.R.4239—Healthy Dog Importation Act, setting a number of requirements for pet owners and veterinarians to adhere to in bringing their dogs into the country. Without a global automated system for tracking and ensuring compliance with these legal requirements it is nearly impossible to implement them effectively.

There is a need for a worldwide digital immunization tracking and authentication system which can be trusted and accessed efficiently by the various parties responsible for pets and livestock. It is within this context that the present invention is provided.

### SUMMARY

The present disclosure provides a system and related methods for automatically recording and managing an immunization record for one or more animals, storing the records on an immutable distributed ledger or blockchain and thus facilitating rapid verification of the status of an animal prior to travel or entry into a restricted area. This allows for immediate on-demand determination of whether pets, wildlife, and livestock are in compliance with the necessary requirements for entry or travel.

Thus, according to one aspect of the present disclosure there is provided a system for recording and managing an immunization record for one or more animals, the system comprising: one or more scanning devices, each configured to read data from a passive transponder or identify a biometric signature of an animal; one or more user devices, each configured to receive identifying data relating to a particular animal from the one or more scanning devices; a distributed ledger database configured to store an immutable record of an immunization history for one or more animals; and one or more servers in communication with the distributed ledger database and the one or more user devices, the one or more servers hosting an online platform for on-demand authentication and updating of an immunization history of one or more animals.

The one or more servers are further configured to: receive an access request from a user device, the access requests comprising identifying information from an animal in the form of an ID number or biometric signature determined by the one or more scanning devices; determine an authorization level of a user account sending the access request; for a first lower authorization level, allow access to an immutable immunization record associated with the ID number or biometric signature as stored on the distributed ledger database; for a second higher authorization level, additionally grant editing permission to the immutable immunization record associated with the ID number or biometric signature as stored on the distributed ledger database.

In some embodiments, the one or more servers are further configured to maintain an updated database of immunization requirements, and in response to the access request, identify and display a relevant set of immunization requirements to the user device.

In such embodiments, the one or more servers may be further configured to determine and indicate to the user device whether the immutable immunization record associated with the animal meets the relevant set of immunization requirements.

In such embodiments, the database of immunization requirements may also comprise one or more sets of legal regulations for entering specific territories with the animal.

In such embodiments, the database of immunization requirements may also comprise one or more sets of transport requirements set by one or more companies responsible for transporting the animal.

In such embodiments, the database of immunization requirements may also comprise one or more sets of entry requirements set by one or more companies for entering a restricted access area.

In some embodiments, if the user account is determined to be of the second higher authorization level, the one or more servers are further configured to receive one or more entries of additional immunizations associated with the animal ID or biometric signature and to update the immutable immunization record with the one or more additional entries.

In some embodiments, the one or more user devices comprise a kiosk having dedicated hardware for interacting with the one or more scanners and one or more servers.

In some embodiments, the one or more user devices comprise a smartphone, tablet, desktop or laptop having dedicated application software installed thereon for interacting with the one or more scanners and one or more servers.

In some embodiments, the one or more scanners comprise one or more RFID readers configured to read an identifying signature from an RFID microchip located under the skin of an animal.

In some embodiments, the one or more scanners comprise one or more biometric readers configured to identify an individual or animal from a fingerprint scan, facial recognition operation, or retina scan.

In some embodiments, the immutable immunization records stored on the distributed ledger each comprise a full immunization history for the associated animal as well as identifying data and relevant associated medical data and/or data identifying the owner.

In some embodiments, only user accounts having been verified as being owned by an accredited and licensed professional or approved by an accredited organisation are granted the second higher authorization level.

In some embodiments, the system utilizes adaptive multi-factor authentication (AMFA) for authentication of the user account sending the access request.

In some embodiments, identifying information of the owner of each animal and/or the user accounts of the platform is managed via smart contracts to maintain privacy.

According to another aspect of the present disclosure there is also provided a use of the system of any one of the above described embodiments to rapidly determine whether an animal meets immunization and health requirements for entry to a specific territory or restricted area by scanning an implanted microchip or biometric signature of the animal.

### BRIEF DESCRIPTION OF THE DRAWINGS

Various embodiments of the invention are disclosed in the following detailed description and accompanying drawings.

FIG. 1 illustrates an exemplary network and hardware architecture over which the methods and system of the present disclosure may be implemented.

FIG. 2 illustrates a flow diagram of an exemplary set of steps which one or more users may carry out according to the present disclosure.

FIG. 3A illustrates a first example interface through which a user may input identity and immunity relevant information regarding one or more animals into a database.

FIG. 3B illustrates a second example interface through which a user may input identity and immunity relevant information regarding one or more animals into a database.

FIG. 3C illustrates a third example interface through which a user may input travel and/or entry information regarding one or more animals into a database.

FIG. 3D illustrates a fourth example interface through which a user may validate identity and immunity relevant information regarding one or more animals by uploading one or more certificates to a database.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The terminology used herein is for the purpose of describing particular embodiments only and is not intended to be limiting of the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present disclosure relates to a system to verify identity and validate immunization history of one or more animals. The system consists of desktop, web and mobile software, blockchain/distributed ledger and biometric and RFID scanning devices.

Instead of manually verifying documentation and making a guess at the correct identity, taxonomy, and health status of an animal, the system allows users to automatically and accurately identify and verify that the animal meets all health requirements for travel and transport. This is achieved by implementing a global network of passive RFID devices, interactive kiosks, servers, desktops, phones and tablets containing smart client and software-as-a-service computer applications using distributed ledger technology to providing licensed and accredited professionals and verified participants and partners instant access to a permissioned single source of truth to help streamline the required processes for in-cabin and cargo travel and transport of pets, livestock and wildlife in a verifiable and permanent way.

The records of pets, livestock and wildlife are uniquely identified by biometric identifiers and/or an integrated circuit (microchip) placed under the skin of the pet, livestock or wildlife entered in by licensed and accredited professionals. Access to the immutable data is granted to a global network of interactive kiosks, servers, desktops, phones and tablets containing smart client and software-as-a-service computer applications to provide participants a single-source of verified truth.

This saves participants such as people and organizations time, money and frustration while reducing the risk for fraud, destruction or loss when using printed documents by providing instant access to verified records in digital format. In addition to managing travel access and requirements, the system can also be used to allow entry into buildings, facilities, etc. much like how proof of vaccination or negative test status for Covid-19 is being required in many work environments.

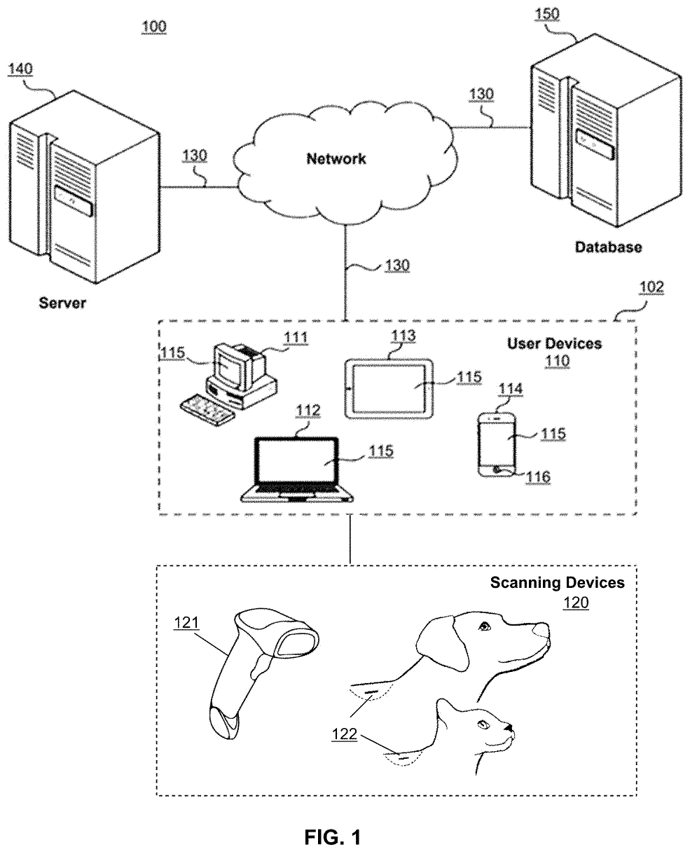

Referring now to FIG. 1, an exemplary embodiment of a immunization record management and verification platform system 100 is shown. In this example, the immunization record management and verification platform system 100 permits a user making use of a user device 110 to securely, rapidly, and automatically view and verify an immunization record of an animal. The platform 100 may communicate with a plurality of user devices 110 of enterprise owners, business owners, and customers to achieve this.

Each user device 110 may share a common user account 102 on the platform, or may be associated with different user accounts. The user account 102 provides for convenient storing and sharing of account information for the same user between user devices 110.

Exemplary user devices 110 as may be used in the immunization record management and verification platform system 100 include, without limitation, a personal computer (PC) 111, a laptop 112, a tablet computer 113, and smartphone 114. Indeed, the user device 110 may be any type of mobile terminal which is regularly carried by an end user and has all the elements necessary for operation in a wireless communication system. The wireless communications include, by way of example and not of limitation, CDMA, WCDMA, GSM, UMTS, or any other wireless communication system such as wireless local area network (WLAN), Wi-Fi or WiMAX.

Generally, each user device 110 includes a display 115 and/or one or more processors. Each of the displays 115 offer the user a visual interface for interaction with the immunization record management and verification platform system 100. For example, the immunization record management and verification platform system 100 may be presented as a browser interface that makes use of known techniques for user interaction.

Each user device 110 is also in communication with a server 140 and database 150 of the system via a network of communications channels 130. The disclosed solution may be beneficially implemented by one or more servers 140 having access to an immutable ledger 114 and communicating with a plurality of client devices 100 over a wireless network architecture. User devices with access to the disclosed system may be permitted to submit requests to view selected portions of verified data stored and verified by the immutable ledger.

The server 140 serves a plurality of user devices 110 having associated user accounts 102, forming a global immunization record system configured to store immunization data relating to one or more animals.

The database 150 may contain user account information, identifying information for one or more animals associated with each user account, account and immunization history, and accredited professional status information and links to various approved organizations.

The database may also comprise a distributed ledger component such as a blockchain, to which users with appropriate levels of authority may upload immutable records of their animals' immunization histories, each associated with a biometric signature or microchip ID number associated with that animal's record.

In this example, the server 140 contains programs facilitating the management of immunization records associated with account shaving different levels of authority, for example, some accounts may be owned by pet owners, livestock owners, and wildlife transporters (referred to herein as “participants”). Such owner accounts may have a lower level of authority, only permitting them to upload identifying information for their animals and view immunization status and history.

Qualified professionals and professional associations such as veterinarians may be given accounts with higher levels of authority, allowing them to upload and validate immunization records for one or more animals to the immutable distributed ledger portion of the database 150.

“Partners” such as airlines, travel booking companies, and security companies needing to manage entry and travel requirements may also be provided with higher levels of authority than owners with access to more information, but are not permitted to edit or add immunization record information for animals.

More particularly it enables healthcare professionals and authorities with the ability to use a microchip scanner and identify the animal and verify it meets all health requirements without having to use documents.

Professionals are required to be accredited and licensed. Data collected include: name, address, city, state, zip, emergency contact information and license and accreditation details.

Smart contracts may be used to handle such identifying information without the need to publicly store it, maintaining privacy or participants.

Participants register with their name, address, city, state, zip, and emergency contact information however will have limited access until they link to an individual or entity from the list of licensed and accredited professionals.

Partners are required to be incorporated entities authorized to do business in their respective jurisdiction. Partners enter their business name, address, city, state, zip, emergency contact information and business incorporation details.

Participants themselves may enter data pertaining to the biometric identifiers and/or passive integrated transponders containing a microchip, but data confirming the identity and history of having specific immnunity against an infectious agent shall not be accessible by partners until being verified by an accredited and licensed professional chosen.

Participants and Professionals can enter travel and transport plans prior to travel and transport. Participants and Professionals that have entered travel and transport plans prior to commencement can instantly access specific in-cabin and cargo requirements, rules and restrictions for having specific immunity against an infectious agent as governed by each respective country, state or local government.

Partners who have been verified will have privileged access to the participant's identity and verification of immunity against an infectious agent which can be used to streamline the in-cabin and cargo travel and transport requirements as mandated by governing entities.

The foregoing are merely illustrative of the architecture of the immunization record management and verification platform system 100 and is not meant to be limiting.

In this example, any user device 110 may include conventional software such as productivity tools (e.g., word processing, spreadsheets, etc.) and at least one browser. Tablet computer 113 or smartphone 114 may also include at least one “app” (defined generally as a purpose-oriented application that may include network communications as part of the functionality).

In some examples, the system may rely on biometric identifiers scanned by a biometric sensor 116 of a user device that can be a conventional optical scanner configured with an appropriate app for use as a fingerprint reader. The fingerprint reader may include software for receiving data from the scanner and interpreting the data within the context of a fingerprint. Other user devices 110 may include a biometric sensor 116 and/or other equipment useful for implementing authentication schemes. Thus, the immunization record management and verification platform system may further implement security measures for securing access to the system.

In addition to biometric scanners for identifying users, a plurality of scanning devices 120 may be incorporated into the system for rapidly and touchlessly identifying animals in order to access and validate their immunization histories as stored on the immutable distributed ledger. For example, RFID scanners 121 may be provided at various terminals and kiosks where validation of animals' immunization histories is required in order to gain access to a restricted area or vehicle for travel to a different territory.

The system can also use scanners 121 in co-ordination with passive integrated transponders containing a microchip 122 for identification. A microchip implant is a passive RFID device which lacks an internal power source and remains inert until it is powered by the RFID scanner or another power source. While the chip itself only interacts with limited frequencies, the device also has an antenna that is optimized for a specific frequency, but is not selective. It may receive, generate current with, and reradiate stray electromagnetic waves. The radio-waves emitted by the scanner 121 activate the chip 122, making the chip transmit the identification number to the scanner, and the scanner displays the number on screen. The microchip is enclosed in a biocompatible glass cylinder and includes an identifying integrated circuit placed under the skin of an mammal. The relevant standards for the chips are ISO 11784, ISO 11785, ISO 14223 and ISO 24631.

The animals would have the microchips 122 implanted subcutaneously, which would respond to the RFID signals of the scanners and allow workers at kiosks and entry points to immediately bring an immunization history of the animal associated with that tag to a display on their user device with a single scan. After data confirming the identity and history of pets, livestock and wildlife having specific immunity against an infectious agent has been entered into the system and verified, users of the system may use an RFID scanner activate the chip, making the chip transmit the identification number to the scanner, and the scanner displays the ID number on screen.

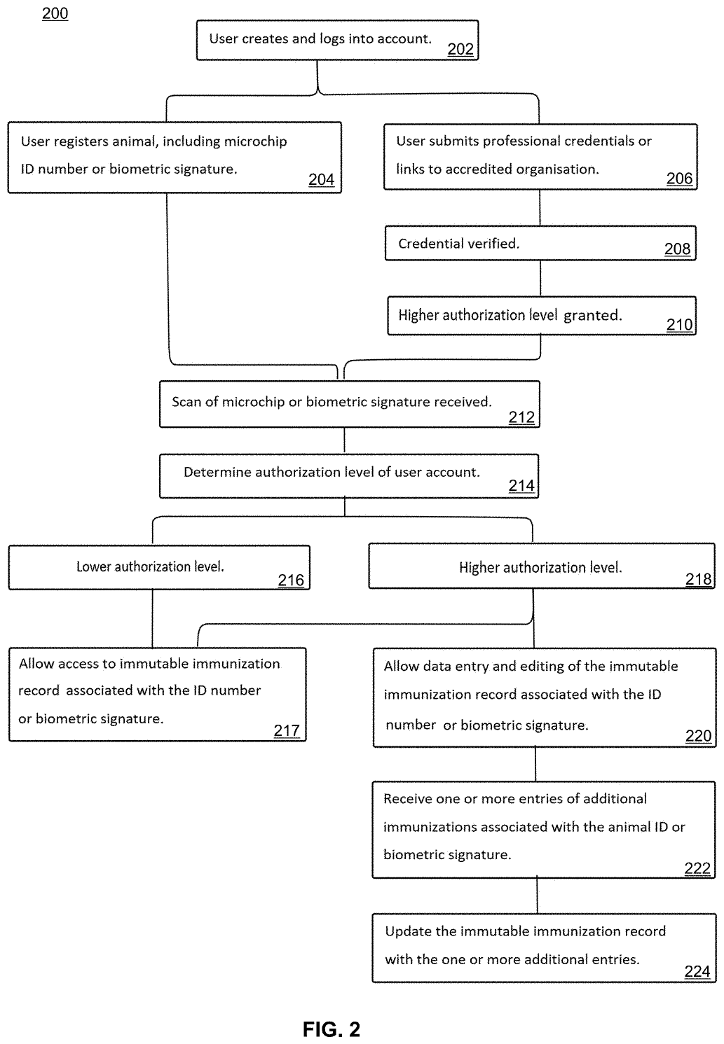

With reference to FIG. 2, the above described processes and operations carried out by accounts having lower or higher authorization levels are shown in steps 202 through 224.

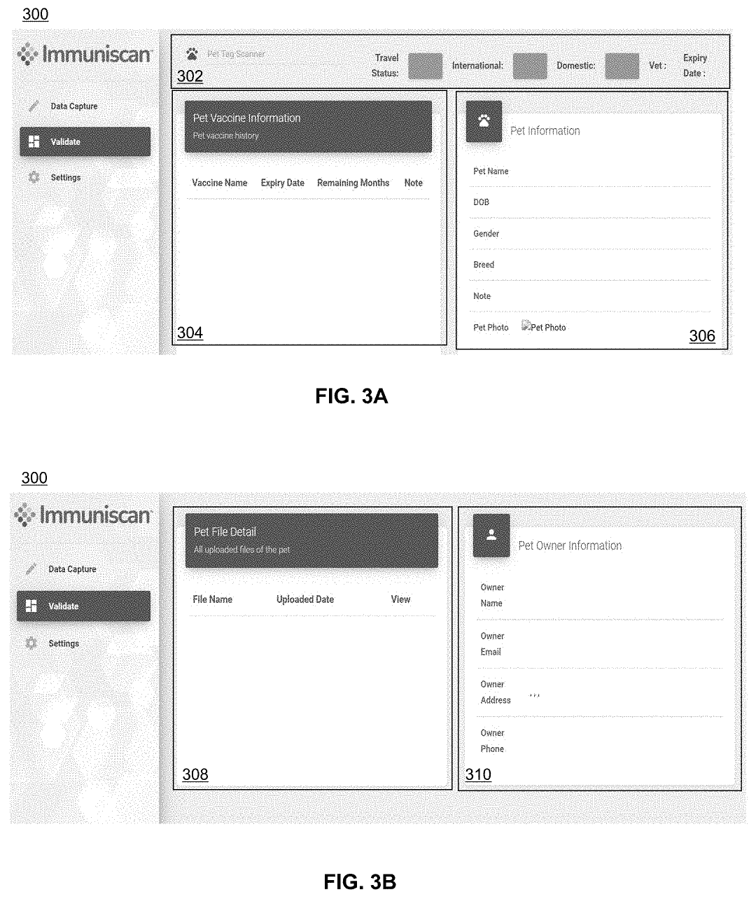

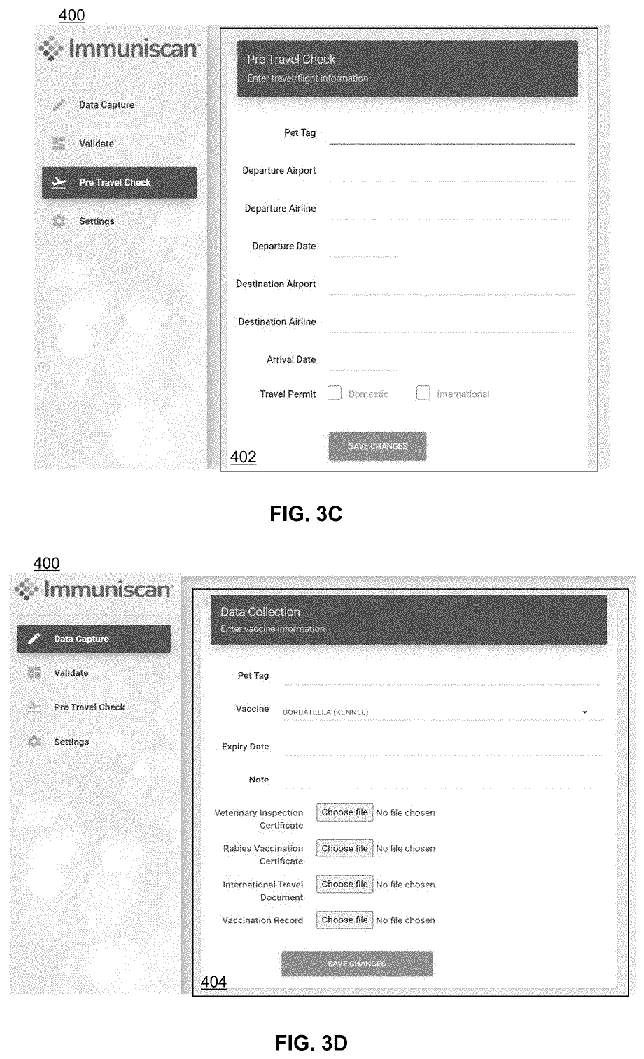

Various interfaces through which identifying and immunization related information may be entered into the system are shown with reference to FIGS. 3A through 3D.

The digitalization of the process also eliminates transcription errors by using the radio-waves emitted by a wired or wireless RFID scanner to instantly activate the microchip, making the chip transmit the identification number to the scanner, automatically displaying the number on the screen of the interactive kiosks, servers, desktops, phones and tablets containing the smart client and access to the software-as-a-service computer applications which automatically query the databases and distributed ledgers to provide professionals, participants and partners with the ability to view specific in-cabin and cargo requirements, rules and restrictions prior to travel and transport and authenticate the identity and verification of immune systems fortified against an infectious agent without the requirement of persistent network connectivity.

It should be understood that the operations described herein may be carried out by any processor. In particular, the operations may be carried out by, but are not limited to, one or more computing environments used to implement the method such as a data center, a cloud computing environment, a dedicated hosting environment, and/or one or more other computing environments in which one or more assets used by the method re implemented; one or more computing systems or computing entities used to implement the method; one or more virtual assets used to implement the method; one or more supervisory or control systems, such as hypervisors, or other monitoring and management systems, used to monitor and control assets and/or components; one or more communications channels for sending and receiving data used to implement the method; one or more access control systems for limiting access to various components, such as firewalls and gateways; one or more traffic and/or routing systems used to direct, control, and/or buffer, data traffic to components, such as routers and switches; one or more communications endpoint proxy systems used to buffer, process, and/or direct data traffic, such as load balancers or buffers; one or more secure communication protocols and/or endpoints used to encrypt/decrypt data, such as Secure Sockets Layer (SSL) protocols, used to implement the method; one or more databases used to store data; one or more internal or external services used to implement the method; one or more backend systems, such as backend servers or other hardware used to process data and implement the method; one or more software systems used to implement the method; and/or any other assets/components in which the method is deployed, implemented, accessed, and run, e.g., operated, as discussed herein, and/or as known in the art at the time of filing, and/or as developed after the time of filing.

As used herein, the terms “computing system”, “computing device”, and “computing entity”, include, but are not limited to, a virtual asset; a server computing system; a workstation; a desktop computing system; a mobile computing system, including, but not limited to, smart phones, portable devices, and/or devices worn or carried by a user; a database system or storage cluster; a switching system; a router; any hardware system; any communications system; any form of proxy system; a gateway system; a firewall system; a load balancing system; or any device, subsystem, or mechanism that includes components that can execute all, or part, of any one of the processes and/or operations as described herein.

As used herein, the terms computing system and computing entity, can denote, but are not limited to, systems made up of multiple: virtual assets; server computing systems; workstations; desktop computing systems; mobile computing systems; database systems or storage clusters; switching systems; routers; hardware systems; communications systems; proxy systems; gateway systems; firewall systems; load balancing systems; or any devices that can be used to perform the processes and/or operations as described herein.

As used herein, the term “computing environment” includes, but is not limited to, a logical or physical grouping of connected or networked computing systems and/or virtual assets using the same infrastructure and systems such as, but not limited to, hardware systems, software systems, and networking/communications systems. Typically, computing environments are either known environments, e.g., “trusted” environments, or unknown, e.g., “untrusted” environments. Typically, trusted computing environments are those where the assets, infrastructure, communication and networking systems, and security systems associated with the computing systems and/or virtual assets making up the trusted computing environment, are either under the control of, or known to, a party.

Unless specifically stated otherwise, as would be apparent from the above discussion, it is appreciated that throughout the above description, discussions utilizing terms such as, but not limited to, “activating”, “accessing”, “adding”, “applying”, “analyzing”, “associating”, “calculating”, “capturing”, “classifying”, “comparing”, “creating”, “defining”, “detecting”, “determining”, “eliminating”, “extracting”, “forwarding”, “generating”, “identifying”, “implementing”, “obtaining”, “processing”, “providing”, “receiving”, “sending”, “storing”, “transferring”, “transforming”, “transmitting”, “using”, etc., refer to the action and process of a computing system or similar electronic device that manipulates and operates on data represented as physical (electronic) quantities within the computing system memories, resisters, caches or other information storage, transmission or display devices.

Those of skill in the art will readily recognize that the algorithms and operations presented herein are not inherently related to any particular computing system, computer architecture, computer or industry standard, or any other specific apparatus. Various general purpose systems may also be used with programs in accordance with the teaching herein, or it may prove more convenient/efficient to construct more specialized apparatuses to perform the required operations described herein. The required structure for a variety of these systems will be apparent to those of skill in the art, along with equivalent variations. In addition, the present invention is not described with reference to any particular programming language and it is appreciated that a variety of programming languages may be used to implement the teachings of the present invention as described herein, and any references to a specific language or languages are provided for illustrative purposes only and for enablement of the contemplated best mode of the invention at the time of filing.

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the system and related methods have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A system for recording and managing an immunization record for one or more animals, the system comprising:
one or more scanning devices, each configured to read data from a passive transponder or identify a biometric signature of an animal;
one or more user devices, each configured to receive identifying data relating to a particular animal from the one or more scanning devices;
a distributed ledger database configured to store an immutable record of an immunization history for one or more animals; and
one or more servers in communication with the distributed ledger database and the one or more user devices, the one or more servers hosting an online platform for on-demand authentication and updating of an immunization history of one or more animals, the one or more servers further being configured to:
receive an access request from a user device, the access requests comprising identifying information from an animal in the form of an ID number or biometric signature determined by the one or more scanning devices;
determine an authorization level of a user account sending the access request;
for a first lower authorization level, allow access to an immutable immunization record associated with the ID number or biometric signature as stored on the distributed ledger database;
for a second higher authorization level, additionally grant editing permission to the immutable immunization record associated with the ID number or biometric signature as stored on the distributed ledger database.

2. A system according to claim 1, wherein the one or more servers are further configured to maintain an updated database of immunization requirements, and in response to the access request, identify and display a relevant set of immunization requirements to the user device.

3. A system according to claim 2, wherein the one or more servers are further configured to determine and indicate to the user device whether the immutable immunization record associated with the animal meets the relevant set of immunization requirements.

4. A system according to claim 2, wherein the database of immunization requirements comprises one or more sets of legal regulations for entering specific territories with the animal.

5. A system according to claim 2, wherein the database of immunization requirements comprises one or more sets of transport requirements set by one or more companies responsible for transporting the animal.

6. A system according to claim 2, wherein the database of immunization requirements comprises one or more sets of entry requirements set by one or more companies for entering a restricted access area.

7. A system according to claim 1, wherein if the user account is determined to be of the second higher authorization level, the one or more servers are further configured to receive one or more entries of additional immunizations associated with the animal ID or biometric signature and to update the immutable immunization record with the one or more additional entries.

8. A system according to claim 1, wherein the one or more user devices comprise a kiosk having dedicated hardware for interacting with the one or more scanners and one or more servers.

9. A system according to claim 1, wherein the one or more user devices comprise a smartphone, tablet, desktop or laptop having dedicated application software installed thereon for interacting with the one or more scanners and one or more servers.

10. A system according to claim 1, wherein the one or more scanners comprise one or more RFID readers configured to read an identifying signature from an RFID microchip located under the skin of an animal.

11. A system according to claim 1, wherein the one or more scanners comprise one or more biometric readers configured to identify an individual or animal from a fingerprint scan, facial recognition operation, or retina scan.

12. A system according to claim 1, wherein the immutable immunization records stored on the distributed ledger each comprise a full immunization history for the associated animal as well as identifying data and relevant associated medical data and/or data identifying the owner.

13. A system according to claim 1, wherein only user accounts having been verified as being owned by an accredited and licensed professional or approved by an accredited organisation are granted the second higher authorization level.

14. A system according to claim 1, wherein the system utilizes adaptive multi-factor authentication (AMFA) for authentication of the user account sending the access request.

15. A system according to claim 1, wherein identifying information of the owner of each animal and/or the user accounts of the platform is managed via smart contracts to maintain privacy.

16. Use of the system of any one of claims 1 to 14 to rapidly determine whether an animal meets immunization and health requirements for entry to a specific territory or restricted area by scanning an implanted microchip or biometric signature of the animal.
